(19) United States
(12) Patent Application Publication
(10) Pub. No.: US 2021/0330830 A1
(43) Pub. Date: Oct. 28, 2021

(54) Ultraviolet Light Germicidal System

(71) Applicant: Keith Zerello, Buchanan, NY (US)

(72) Inventor: Keith Zerello, Buchanan, NY (US)

(21) Appl. No.: 16/901,063

(22) Filed: Jun. 15, 2020

Related U.S. Application Data
(60) Provisional application No. 63/014,359, filed on Apr. 23, 2020.

(51) Int. Cl. A61L 2/10

(52) U.S. Cl. CPC A61L 2/10; A61L 2202/16; A61L 2202/23; A61L 2202/14; A61L 2202/11; A61L 2202/121

(57) ABSTRACT

This present invention relates to an ultraviolet (UV) based germicidal system which can be utilized as a cleansing system to sterilize commonly touched items or surfaces with ultraviolet radiation. The system is comprised of a UV light assembly that can be integrated into the manufacturing stage of a specific item, or installed on existing items as an aftermarket product. The system also includes a power source, such as direct wiring, a battery, or solar panels; one or more UVC bulbs; a safety contact switch to deactivate the UVC bulb or bulbs; and a timer to conserve power and bulb longevity.

### CROSS-REFERENCE TO RELATED APPLICATION

The present application claims priority to, and the benefit of, U.S. Provisional Application No. 63/014,359, which was filed on Apr. 23, 2020 and is incorporated herein by reference in its entirety.

### BACKGROUND

The present invention relates generally to the field of ultraviolet radiation, and more particularly, to a device including one or more ultraviolet emitters mounted thereto for disinfecting surfaces, articles and the like. More specifically, the present invention relates to a novel and improved method and apparatus for the ultraviolet radiation-based disinfection and sterilization of high touch surfaces such as mailboxes, cash drawers, ATMs, or other items. The ultraviolet cleansing system of the present invention assists in reducing viruses, bacteria, and other pathogens present on surfaces that may cause transmission of diseases to individuals. Accordingly, the present specification makes specific reference thereto. However, it is to be appreciated that aspects of the present invention are also equally amenable to other like applications, devices and methods of manufacture.

Personal hygiene, as well as public hygiene, play an important role in society and the daily life of each of its individuals. Maintaining good hygiene by washing hands frequently and cleaning surfaces regularly can aid in preventing transmission of many pathogen borne illnesses from infected surfaces. Because such microbes are not visible to the human eye, it is always a concern whether a surface or article is free of illness causing microorganisms.

Various communicable and pandemic diseases are spread to individuals when a person comes into contact with a contaminated surface. For example, pathogenic microbes such as bacteria, viruses, fungi, molds and other harmful microorganisms are easily transmitted by touching or using everyday objects, such as mail, money, gas pumps, garbage cans, public transport, gym and the like. Once an infected individual touches an infected surface, the individual leaves microbes and their spores on the surface and other individuals are also at risk to acquire infection. The elderly or those with compromised immune systems, such as people suffering from HIV, diabetes, or other immune suppressing illness, are at an increased risk of becoming infected.

By the way of background, disinfectants are often used to sterilize surfaces. However, such disinfectants may be manufactured using harsh chemicals including, without limitation, alcohol, chlorine and chlorine compounds, formaldehyde, glutaraldehyde, iodophors, ortho-phthalaldehyde (OPA) Peracetic Acid, peracetic acid, hydrogen peroxide, phenolics, etc. Arguably, it is not safe to use such hazardous chemicals to prevent the spread of germs and other pathogens, as regular or accidental ingestion or inhalation of such harmful chemical substances can be detrimental to one's health. Additionally, the public oftentimes demands sterilization methods which are safe, effective and that do not have a detrimental impact on the environment or the individual using the same.

Existing approaches to disinfecting enclosed structures, such as mailboxes, garbage bins, cash drawers, etc. have included the use of sterilizing liquids and/or gases. Sterilizing liquids may include bleach or stronger chemicals, which may have a detrimental, or, at the very least, a discoloring effect on the surfaces or materials to which the liquids are applied. In addition, sterilizing liquids oftentimes may not be applied safely to electronics or other devices that receive electrical power. Furthermore, liquids when applied to absorbent or fragile substances or materials, such as paper, may lead to said substances or materials becoming damaged.

Similarly, disinfecting or fumigating gases may include harsh chemicals that are toxic to both individuals and animals. Therefore, in such scenarios, said individuals and animals must be safely removed from the areas where fumigating gases are being used. Additionally, a significant amount of time is typically required for the toxic gas used to sufficiently dissipate from the area once the fumigating is complete. Unfortunately, this amount of time may be many hours or even days, which is highly inefficient.

Further, the existing methods of disinfecting liquids and gases are applied manually to a surface or an area and if the user is not knowledgeable about the same or does not take appropriate precautions, the user may experience health problems such as respiratory issues, skin deterioration, rashes, etc. Additionally, existing disinfecting liquids and gases are not always controllable based on the surface requirements, germ status, environment requirements, human and animal safety, etc. Neither of the existing approaches is desirable as they utilize unsafe chemicals, damage absorbent or fragile substances, introduce toxic agents in the environment, require prolonged period of use, have to be applied manually, and require substantial knowledge and precautions by the user to avoid adverse outcomes.

Therefore, there exists a long felt need in the art for a safe yet effective sterilization method which does not include the use of harsh chemicals, and a convenient method to clean surfaces of daily use articles which are a medium for transmitting pathogens. There is also a long felt need in the art for disinfecting methods that are applicable to absorbent and fragile substances and sterilizing methods that makes the disinfected area quickly accessible to the user. Further, there is also a long felt need in the art for automatically operable and controllable cleansing methods, and for a portable disinfectant device. Finally, there is a long felt need in the art for a sterilization system which can be easily controlled by the user.

The subject matter disclosed and claimed herein comprises an ultraviolet radiation based germicidal source such as a lamp, which is a cleansing system designed to assist in reducing and possibly eliminating viruses, bacteria, and other pathogens from commonly touched items, such as mails/mailboxes, cash drawers, ATMs, and other highly touchable surfaces. The invention, in one embodiment, is comprised of a UV light assembly that can be either integrated in the manufacturing stage of a specific item, or can be installed later as an aftermarket item. The light UV light assembly is comprised of a power source, one or more UVC bulbs, a safety contact switch to turn on/off the sterilization system, and a timer to conserve power and bulb longevity. The size of the assembly may vary based on the particular application or the user's needs and/or preferences. The UV light-based cleansing system is designed to sterilize the area illuminated by the plurality of UVC bulbs.

In this manner, the ultraviolet radiation based germicidal lamp of the present invention accomplishes all of the forgoing objectives, and provides a relatively quick and convenient solution for sterilizing commonly touched surfaces or items, such as mail/mailboxes, cash drawers, ATMs, and more. The improved ultraviolet radiation based germicidal lamp of the present invention is also safe, portable and easy to install, thereby reducing costs and improving overall sterilizing efficiencies illustrating a benefit of the present invention. Finally, the improved ultraviolet radiation based germicidal lamp of the present invention can be used to specifically sterilize surfaces which are prone to carry pathogenic microbes.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises an ultraviolet light germicidal system for use with objects, articles and surfaces such as a mailboxes, cash drawers, ATMs, portable bags, garbage bins or other similar type items which are prone to carry germs and assist in reducing and possibly eliminating viruses, bacteria, fungi, molds and other pathogens. The invention also prevents individuals, especially the immuno-compromised, from contracting an illness or disease when partaking in everyday activities, such as retrieving the mail, and helps to prevent the spread of not only viruses and bacteria, but also mold, fungus, and undesirable odors. The invention eliminates the need for use of harsh or toxic chemicals to kill pathogens, and the adverse consequences of the same.

The present invention is an ultraviolet radiation sterilization system that is preferably comprised of at least one UV or UVC bulb, an internal power source, a switch, a timer and a controller, and may be affixed to a substrate of an object. More specifically, in one embodiment of the present invention, an ultraviolet radiation based germicidal light source, such as a lamp, is fixed to a surface of a mailbox for sterilizing mail items such as postcards, envelopes, brochures, cds, and so forth, kept inside the mailboxes, by illuminating the antimicrobial ultraviolet light over a contaminated area of the mailbox. Structurally, the invention is comprised of a UV light assembly that can be integrated into the manufacturing stage of a specific item or installed on existing items. The system also includes a power source such as direct wiring, a battery, or solar panels; one or more UVC bulbs depending on the size of the space to be sterilized; a safety contact switch to deactivate the UVC bulbs, for example, when the mailbox door is opened; and a timer to conserve power and bulb longevity. The size of the assembly may vary based on the specific application and/or the wants and needs of the user.

In yet another embodiment of the present invention, an ultraviolet radiation based germicidal lamp is attached to a cash drawer. More specifically, the UV light disinfecting assembly is preferably placed on an inner side of the top surface of the drawer housing, such that when the cash drawer is closed, the UV based germicidal lamp illuminates the cash drawer area and disinfects the items such as cash, coins, etc. kept inside the drawer. The UV light disinfecting assembly includes a power source, a controller and a battery to power the UVC bulbs. In a further embodiment, the UV light disinfecting assembly also comprises a timer for automatically configuring the disinfection of the items for a required time period, thereby conserving power and increasing the useful life of the UVC bulbs. Further, the UV light disinfecting assembly may also comprise a plurality of sensors for sensing or detecting the presence of germs, open drawer status, etc.

In yet another embodiment of the present invention, an ultraviolet radiation based germicidal lamp with automatic UVC germicidal function is attached to an ATM machine. For example, a keypad of the ATM machine is a heavily trafficked surface and typically contains pathogenic bacteria and viruses which may cause disease in individuals who come in contact with the same. The UV lamp assembly is fixed to a surface of the ATM machine, such that the UV light illuminates the keypad area, or any other desired location on the ATM machine, to disinfect the same.

In yet another embodiment of the present invention, an ultraviolet radiation based germicidal lamp with automatic UVC germicidal function is attached to a portable bag. Household articles, smartphones, tablets, kitchen utensils, washing tools, baby supplies, pacifiers, toys etc. usually contain pathogenic bacteria and viruses which cause diseases in individuals who come in contact with such items. The UV lamp assembly is fixed to a portable bag, such that the UV light illuminates the articles present in the bag, thereby sterilizing the articles which are often touched by different users and preventing said individuals from coming into contact with various communicable diseases.

In still yet another embodiment of the present invention, an ultraviolet radiation sterilization system for a drawer is provided that comprises a drawer having an interior chamber where ultraviolet radiation occurs, at least one UV-C or UV-C bulb or tube, a plurality of sensors to detect if said drawer is in an open or closed position, and a power wire leading to a power supply, wherein said system is triggered and ultraviolet illumination occurs when said drawer is in the closed position.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one possible embodiment of an ultraviolet radiation based germicidal lamp system of the present invention affixed to a surface of a mailbox in accordance with the disclosed architecture.

FIG. 2 illustrates a perspective view of one possible embodiment of a switching system assembled with the germicidal lamp system of FIG. 1 to activate and/or deactivate the UV lights based on the mailbox door condition in accordance with the disclosed architecture.

FIG. 3 illustrates a perspective view of one possible embodiment of components of a solar power assembly for powering the germicidal lamp system of FIG. 1 in accordance with the disclosed architecture.

FIG. 4 illustrates a perspective view of one possible embodiment of a cash drawer germicidal lamp system of the present invention in accordance with the disclosed architecture.

FIG. 5 illustrates a perspective view of one possible embodiment of an ATM germicidal lamp system of the present invention in accordance with the disclosed architecture.

FIG. 6 illustrates a perspective view of one possible embodiment of a portable UV-C Light UV sterilization bag in accordance with the disclosed architecture.

FIG. 7A illustrates a perspective view of one possible embodiment of a portable UV-C light assembly of the present invention in accordance with the disclosed architecture.

FIG. 7B illustrates a one possible embodiment of a circuit diagram of the portable UV-C light assembly of FIG. 7A in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there exists a long felt need in the art for a safe yet effective sterilization system and method, which does not include the use of harsh chemicals. There is also a long felt need in the art for a convenient system and method for cleaning surfaces of daily use articles which can be a medium for transmitting pathogens, and for disinfecting systems and methods that are applicable to absorbent and fragile articles and substances. Additionally, there is a long felt need in the art for sterilizing systems and methods that make the disinfected area quickly accessible to the user, as well as for automatically operable and controllable cleansing methods. Finally, there is also a long felt need in the art for a portable disinfectant device that is easily controllable and regulated by the user.

The innovative product of the present invention features an ultraviolet radiation germicidal system for use in or on surfaces and substrates, objects, and articles, such as mailboxes, cash drawers, ATMs, lockers, and other item, which are prone to carry and transmit germs, bacteria, diseases and the like. The UV or UVC or far-UVC based cleansing system of the present invention assists users in sterilizing and possibly eliminating viruses, bacteria, fungi, molds and other pathogens from such objects and places to reduce transmission of microbes as well as diseases caused by such pathogens. The invention also prevents individuals, especially those who are immuno-compromised, from contracting an illness or disease when partaking in everyday activities, such as retrieving mail, and helps to prevent the spread of not only viruses and bacteria, but also mold, fungis, odors, and the like. The invention eliminates the need for the use of harsh or toxic chemicals to kill pathogens.

The present invention, in one exemplary embodiment, is an ultraviolet radiation cleansing system comprising one or more UVC/far UVC lamps or bulbs for illuminating antimicrobial ultraviolet radiation in a contaminated area and sterilizing germ prone items. The system is further comprised of a power source, such as direct wiring, a battery, and/or solar panels, for powering the UV of UVC bulbs; a safety contact switch to activate and/or deactivate the UV or UVC bulbs; and a timer for controlling the duration that the UV or UVC bulbs remain powered to conserve power and bulb longevity. The present invention also contemplates that the system may comprise a combination of power sources, and is not limited to just one particular source of power. Further, the number and size of the UV or UVC bulbs may vary based on the particular application, or the wants and needs of the user. The cleansing system is applicable to various devices including, without limitation, mailboxes, ATMs, cash drawers, gas pumps, garbage cans and other frequently contaminated areas.

In a preferred embodiment, the ultraviolet radiation based germicidal lamp assembly of the present invention is affixed to a surface of a substrate. For instance, in one embodiment, the ultraviolet radiation system is attached to an interior surface of a mailbox for sterilizing mail items such as postcards, envelopes, brochures, etc., placed inside the mailbox, by illuminating the antimicrobial ultraviolet light over a contaminated area of the mailbox. Structurally, the sterilizing UV light system of the present invention is comprised of a UV light assembly that can be integrated into the manufacturing stage of a specific item or installed on an existing item as an aftermarket add-on. The system also includes a power source such as direct wiring, a battery, and/or solar panels; one or more UVC bulbs depending on space and size requirements and limitations; a safety contact switch to deactivate the UVC bulbs, for example, when the mailbox door is opened; and a timer to conserve power and the longevity of the UVC bulbs.

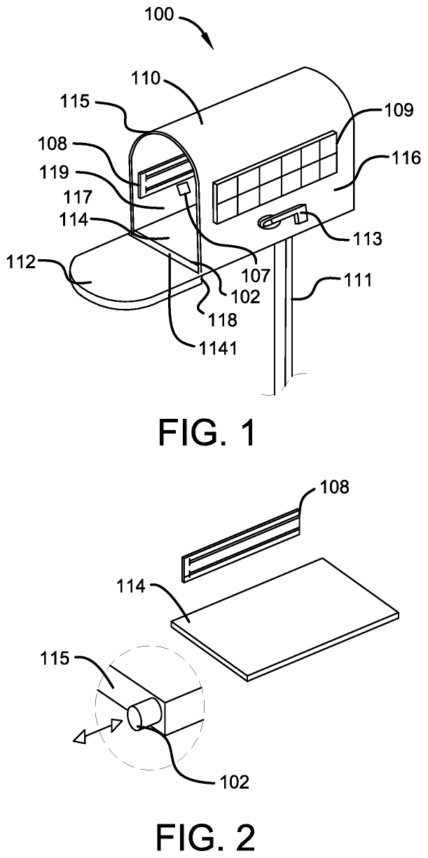

Referring initially to the drawings, FIG. 1 illustrates a perspective view of one possible embodiment of an ultraviolet radiation based system 100 having a germicidal lamp assembly 108 affixed to a surface of a mailbox 110 of the present invention in accordance with the disclosed architecture. More specifically, the mailbox germicidal lamp system 100 is comprised of mailbox 110 having a conventional shape and comprising a mailbox door 112, a mailbox insert 114, a mailbox insert front edge 1141, a mailbox right wall 116, a mailbox left wall 117, and a mailbox interior chamber 119. The mailbox door 112 is pivotally attached by a hinge member 118 to an open end 115 of the mailbox 110. In a conventional format, the mailbox 110 is mounted on a post 111, and may further comprise a flag 113 that can be raised to indicate to a postal worker or resident that items are inside the mailbox 110 for pickup or retrieval.

Mailboxes 110 are containers that are useful for receiving and housing mail related items, such as postcards, envelopes, brochures, packages and the like. Unfortunately, such mail items are prone to carrying microbes, bacteria, germs and the like that may infect a person coming in contact with the mail items of the mailbox 110 in which they are received. UV lamp assembly 108 can be helpful in eliminating or at least reducing the presence of such bacteria, viruses, germs and the like from the mail items and mailbox interior chamber 119 by illuminating UV light and radiation on the same while the mailbox 110 is not in use (i.e., the mailbox door 112 is in the closed position).

More specifically, the mailbox or container 110 includes one or more ultraviolet light bulbs 108 attached to the right or left wall surfaces 116, 117 of the mailbox 110. However, the placement of the ultraviolet light bulbs 108 is not limited, and can be placed on any other interior surface of the mailbox 110 as well. The ultraviolet light bulbs 108 are preferably UV germicidal bulbs that emit relatively short ultraviolet wavelengths, for example from 100 to 280 nanometers, that damage the DNA of bacteria, viruses, and other pathogens. The use of such ultraviolet light bulbs 108 can help eradicate harmful bacteria, and can be used effectively for a variety of places where sterilization is needed, such as mailboxes, cash drawers, ATMs, and/or in the purification of food, water and air. The ultraviolet light bulbs 108 are designed to sterilize the area in which they illuminate. In a preferred embodiment, the ultraviolet light bulbs 108 can be selected from either a UVC or a far-UVC bulb. Far-UVC ultraviolet light bulbs 108 have a shorter wavelength, a higher frequency, and a higher energy than UVC light bulbs, thereby making them more effective on undesirable microorganisms.

In a preferred embodiment, the mailbox germicidal lamp system 100 further comprises a power source such as a battery (not shown) that may be charged by a solar panel 109 attached to the exterior surface of the mailbox 110. More specifically, the battery may be used to power the ultraviolet light bulb assembly 108 to emit ultraviolet radiation when required for disinfecting the insert surface 114 and when traditional hardwired electrical power is not readily available. Further, the system 100 includes a safety contact switch 102 that may be provided on the mailbox insert front edge 1141. The system 100 includes a controller (not shown) and a timer 107 for automatic operation of the UV bulb assembly 108 and disinfection of the mailbox 110, and the system 100 operates automatically once the mailbox door 112 is in the closed position. More specifically, when the mailbox door 112 is in a closed position, the door 112 pushes the safety contact switch 102 which, in turn, signals the controller to turn on the UV light assembly 108. The UV light assembly 108 coordinates with the timer 107 and operates for a predetermined time period to sterilize the interior chamber 119 of the mailbox 110. The predetermined time period may be selected by a user (not shown) to satisfy user need and/or preference or to conform to a specific safety and/or disinfection standard. Additionally, when the mailbox door 112 is in an open position (e.g., to enable a user to retrieve the contents of the mailbox), the mailbox door 112 releases the safety contact switch 102, which signals the controller to automatically turn off the UV light assembly 108, thereby conserving electrical power/battery life and increasing the useful life span of the ultraviolet light bulbs 108.

In a further preferred embodiment of the present invention, the mailbox germicidal lamp assembly 100 may also comprise a plurality of sensors such as pressure sensors, motion sensors, door condition sensors, germ detection sensors, etc. (not shown). For example, a pressure sensor can be used to monitor the mailbox interior pressure, and shut down the radiation source if a dangerous pressure such as 5 atmospheres or more exists. The door condition sensor may detect if the mailbox door 112 is in an opened or closed position and accordingly activate or deactivate the ultraviolet light bulb assembly 108 to conserve power and extend bulb life, as well as reduce exposure of the user to the UV light.

Similarly, the proximity of motion sensors may be used to switch off the ultraviolet light bulb assembly 108 when a user or mail delivery person is in a user selectable proximity to the mailbox 110 to receive or dispense the mail items. Further, the germ sensors may be used to detect the presence of germs, bacteria, mold, etc., in the mailbox interior chamber 119 (e.g., on the mail items) and accordingly regulate the sterilization cycle for the mailbox 110.

FIG. 2 illustrates a perspective view of a switching system assembled with a mailbox germicidal lamp to activate and/or deactivate the UV lights based on the mailbox door condition in accordance with the disclosed architecture. As previously mentioned, the switching system 102 may be attached to a front edge 1141 of mailbox insert 114 of mailbox 110, and is used to activate or deactivate the ultraviolet light bulb assembly 108. The safety contact switch 102 is turned on when the mailbox 110 is not in use and the mailbox door 112 is in a closed condition. More specifically, the mailbox door 112 pushes or applies pressure to the safety contact switch 102, thereby signaling the controller (not shown) to turn on the UV light assembly 108 within the mailbox 110. Likewise, the safety contact switch 102 is automatically released when the mailbox door 112 is in an open position, thereby deactivating the ultraviolet light bulb assembly 108 and saving electrical power. Additionally, the automatic operation of the safety contact switch 102 based on the condition or positioning of the mailbox door 112 increases the life span of the UVC bulbs 108. Further, the safety contact switch 102 may also be placed on any outer surface of the mailbox 110 to allow the user to manually activate or deactivate the UV disinfection system 100, regardless of the position of the mailbox door 112.

In an embodiment of the present invention, automatic operation of the mailbox germicidal lamp system 100 is enabled using sensor mechanisms that turn ON or turn OFF the ultraviolet light bulbs 108 based on data collected by various incorporated sensors (not shown). For example, a sensor, such as a proximity sensor, may be used to detect the presence of a person and/or movement nearby a mailbox 112 and, accordingly, deactivate the system 100. Another sensor, such as a pressure sensor, can be used to monitor the mailbox interior pressure and shut down the ultraviolet light bulbs 108 if a dangerous pressure, such as 5 atmospheres or more, exists. Furthermore, a door condition sensor could be used to detect if the mailbox door 112 is in a closed or opened position and, accordingly, activate or deactivate the ultraviolet light bulbs 108.

In a further embodiment of the present invention, the mailbox 110 may further comprise a warning light indicator and/or a safety light indicator (not shown). For example, the warning light indicator may notify the user not to open the mailbox 110, and the safety indicator may notify the user that the mailbox 110 is safe to use. Both the warning and safety light indicators can be any LED light indicators of multiple colors or the like. Additionally, the mailbox 110 may also comprise a plurality of air vents, an air circulating system and a fan that are utilized to cool the interior of the mailbox 110 with outside ambient air after the decontamination cycle is complete. Further, the mailbox 110 may also include a thermometer to determine if the mailbox 110 and the mail items contained therein have cooled down sufficiently, and signal the controller to indicate to the user (e.g., through the above referenced safety light indicator) that it is safe to open the mailbox 110.

In a further embodiment of the present invention, the interior walls of the mailbox 110 of the mailbox germicidal lamp assembly 100 of the present invention may be coated with a galvanized paint to enhance the reflective properties of the mailbox material and illumination of the ultraviolet light within the mailbox interior, thereby improving the efficiency of the system 100. Inasmuch as a common abundance of germs, bacteria, mold and the like may be found on the outside surface of most mail items, especially those being recently handled, the interaction of the ultraviolet light reflecting from a plurality of inner surfaces of the mailbox should be sufficient for most applications.

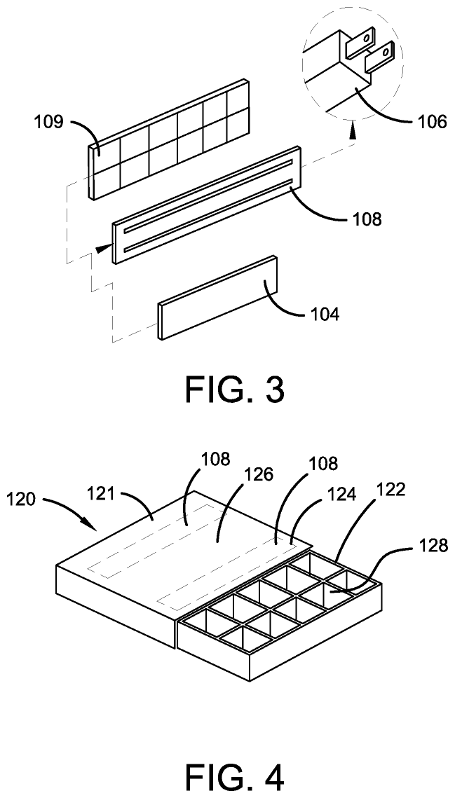

FIG. 3 illustrates a perspective view of basic components of the mailbox germicidal assembly such as an internal battery 104, a power system 106, one or more ultraviolet light bulbs 108 and an exterior solar panel 109. Battery 104 may be any suitable battery for powering the assembly including, without limitation, AA batteries, an M battery, a solar-powered battery, or a lithium battery. Access to the internal battery 104 can be provided by any number of conventional means, such as a threaded plug, a sliding cover etc. Additionally, the power system 106 can be a power inverter or DC power source.

FIG. 4 illustrates a perspective view of one potential embodiment of a cash drawer germicidal light source in accordance with the disclosed architecture. The drawer germicidal lamp 120 preferably includes one or more UVC or far-UVC bulbs 108, and may be attached to a cash drawer 121 that is comprised of a cash tray 122 containing various receptacles for receiving currency and/or coin, an outer box 124 for receipt of cash tray 122 and for providing structural support and protection thereto, a top surface 126 and a cash drawer interior chamber 128, where the ultraviolet illumination occurs when the cash tray 122 is in a closed condition (e.g., within the outer box 124). While the present invention discusses that the system of the present invention being utilized with a cash drawer 121 for the sterilization of currency contained within the cash tray 122, it is important to note that the system of the present invention is not limited to the same and may also be used with any type of drawer presently contemplated such as, without limitation, a clothes drawer, a kitchen drawer, a utility drawer, etc.

In one embodiment of the present invention, the cash drawer germicidal lamp 120 may also comprise a plurality of sensors such as pressure sensors, motion sensors and the like to detect if the cash tray 122 is in an opened or closed position. More specifically, the cash drawer germicidal lamp 120 is preferably triggered upon the opening and closing of the cash tray 122. For example, the pressure sensor may monitor the cash drawer's interior pressure, and switch off the germicidal lamp 120 if the cash tray 122 is determined to be opened. As another example, a motion sensor helps in switching off the UVC or far-UVC bulbs 108 when a user is nearby the cash drawer 121 to retrieve or deposit currency into the tray 122. Further, the cash drawer germicidal lamp 120 may comprise a power wire and a corresponding lead to a power supply, to power the UVC or far-UVC bulbs 108.

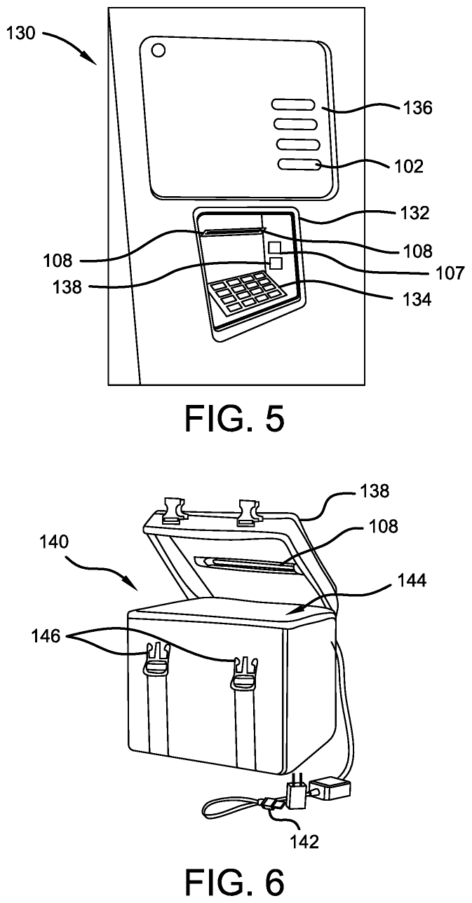

FIG. 5 illustrates a perspective view of one possible embodiment of a UVC germicidal lamp of the present invention used on a cash dispensing ATM machine 130 having a touch key unit 134 in accordance with the disclosed architecture. The UVC germicidal lamp for ATM machine 130 preferably includes one or more UVC or far-UVC light bulbs 108, a UV light switch 102, a controller 138, and a timer 107. The UVC light bulbs 108 sanitize the tactile input mechanisms of the ATM, such as the touch key unit 134, from bacteria, viruses, germs and other microorganisms that are easily transferred from one user of the ATM machine 130 to a subsequent user. In a preferred embodiment of the present invention, the controller 138 will activate the UV germicidal bulbs 108 for a minimal amount of time which may be established through the timer 107 after each use of the ATM machine 130. After each sterilization, the sterilized touch key unit 134 of the ATM machine 130 is ready for use by the next customer.

In an alternative embodiment, the activation of the UVC germicidal lamp 108 may be determined according to a particular time of day, as may be selected to satisfy user need and/or preference. For example, an ATM inside of a bank may be programmed to activate the UVC germicidal lamp 108 during hours in which the bank is closed to the public, thereby ensuring adequate sterilization at least before and after banking hours, while minimizing or eliminating potential exposure of the UVC germicidal lamp 108 to the public. Alternatively, the intensity or wavelength of the UVC germicidal lamp 108 may be minimized during periods of frequent use, as detected by the various sensors described above, such as motion and/or proximity sensors. However, during periods of decreased or infrequent usage, the controller 138 may increase the intensity or wavelength of the UVC germicidal lamp 108 by altering the power input to the UV light source 108. The embodiment of the UVC germicidal lamp 108 for ATM machine 130 may also utilize a UV protective shield (not shown) to protect users from UV radiation exposure while in use. For example, and not by way of limitation, the UV protective shield may be a physical cover that is comprised of an UV impermeable material to block UV radiation from contacting the user.

FIG. 6 illustrates a perspective view of portable UV-C light sterilization bag in accordance with the disclosed architecture. The portable UV-C light sterilization bag 140 of the present invention preferably comprises of USB input or port 142 for charging the sterilization bag 140, a bag interior space 144, one or more securing fasteners 146, a bag lid or closure 148, one or more UVC or far-UVC bulbs (or tubes) 108 present on the inner surface of the bag lid 148 to provide the ultraviolet light necessary for the proper sterilization of the bag interior space 144 and the contents contained therein. More specifically, the UV-C light sterilization bag 140 uses high-precision special wavelength ultraviolet lights 108 to sterilize and disinfect the surfaces of the articles placed in the bag interior space 144. A controller (not shown) may be used to switch “on” the UVC bulbs 108 once the securing fasteners 146 are fastened, and to switch “off” the UVC bulbs 108 once the securing fasteners 146 are unfastened. Other fastening mechanisms such as a zipper may also be used which, once completely zipped, may switch “on” the high-precision special wavelength ultraviolet light UVC 108 to initiate the sterilization. The portable UV-C Light sterilization bag 140 of the present invention may be used to sterilize, for example, household items, kitchen utensils, tools, baby supplies, pacifiers, toys, etc., and may further comprise a timer (not shown) for programming when and for how long the UVC or far UVC bulbs 108 will remain switched on. The UVC or far-UVC bulbs 108 of the present invention are preferably both mercury and ozone free, and may be comprised of LED beads. Additionally, the UVC or far-UVC bulbs 108 may be powered by one or more batteries, and may be manually switched on or off by a user using a switch 102 as described above.

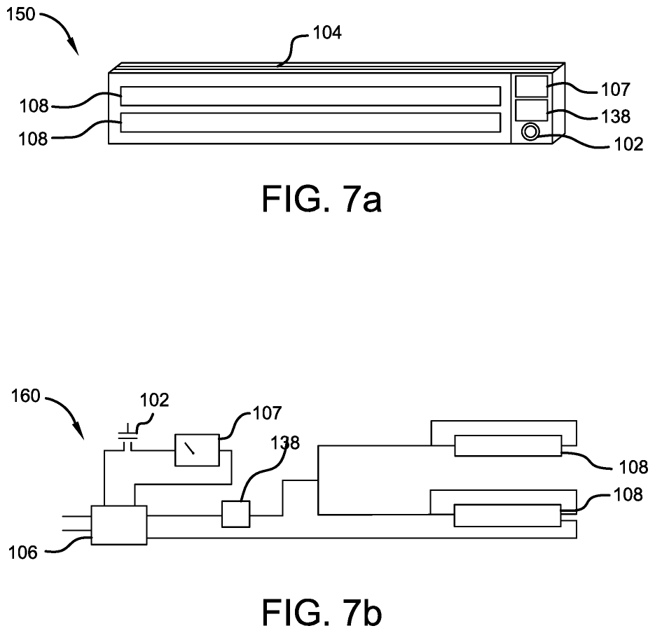

FIG. 7A illustrates a perspective view of one possible embodiment of a portable UV-C light assembly of the present invention. More specifically, a standalone UV based germicidal product 150 comprises at least one UVC lamp or bulb 108 for illuminating ultraviolet light/radiation over a germ prone surface, object, item or the like. The germicidal product 150 includes an internal power source, such as a battery 104 positioned on the portion of the product 150 to power the UV-C lamps 108. The internal battery 104 is preferably rechargeable, and may be powered by any external power source, direct wiring and/or solar panels. The standalone UV based germicidal product 150 further comprises a switch or a button 102 to turn “on” or “off” the UV-C lamps 108, a timer 107 to conserve power and bulb longevity, and a controller 138 to manage operation of the product 150 and its various components.

FIG. 7B illustrates one possible embodiment of a circuit diagram 160 of the portable UV-C light assembly 150 of FIG. 7A in accordance with the disclosed architecture. The circuit diagram comprises a power system 106 that supplies power to the UV-C lamps 108. Upon opening of the switch 102, the timer is energized and allows the power supply 106 to power the battery 104 (not shown) to deliver the power to the UV-C lamps 108 for a user selectable period of time. Further, the controller 138 manages operation of the UV-C lamps 108 and other components.

It is to be understood that the system of the present invention is not limited in its application to the details of construction and to the arrangements of the components set forth above. Rather, the system of the present invention is capable of being practiced and carried out in various ways and in a variety of different embodiments. Also, it is to be understood that the phraseology and terminology employed herein are for the purpose of description and should not be regarded as limiting.

Certain terms are used throughout the following description and claims to refer to particular features or components. As one skilled in the art will appreciate, different persons may refer to the same feature or component by different names. This document does not intend to distinguish between components or features that differ in name but not in structure or function. As used herein “microbes”, “pathogens”, “microorganisms”, and “germs” are interchangeable and refer to the disease-causing bacteria, viruses, fungi and molds. The terms “sterilization” and “cleansing” also hold the same meaning as of the removal of bacteria, viruses, fungi and molds from any object and can be used interchangeably. As used herein “far UVC bulbs”, “UVC Bulbs”, “ultraviolet tubes”, “UVC tubes”, “far UVC tubes”, “germicidal lamps”, “ultraviolet light bulbs” and “UV lights” are interchangeable and refer to the disinfecting and sterilizing UVC or far-UVC or UV light.

Notwithstanding the forgoing, the germicidal lamp system 100, 120, 130 and 140 of the present invention and its various structural components can be any suitable size, shape, and configuration as is known in the art without affecting the overall concept of the invention, provided that it accomplishes the above stated objectives. One of ordinary skill in the art will appreciate that the shape and size of the germicidal lamp system 100, 120, 130 and 140 and its various components, as shown in the FIGS. are for illustrative purposes only, and that many other shapes and sizes of the germicidal lamp system 100, 120, 130 and 140 are well within the scope of the present disclosure. Although dimensions of the germicidal lamp system 100, 120, 130 and 140 and its components (i.e., length, width, and height) are important design parameters for good performance, the germicidal lamp system 100, 120, 130 and 140 and its components may be any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An ultraviolet radiation sterilization system comprising:
at least one bulb;
an internal power source;
a switch;
a timer and a controller; and
wherein said ultraviolet radiation sterilization system may be affixed to a substrate.

2. The sterilization system of claim 1, wherein said system is portable.

3. The sterilization system of claim 1, wherein said power source is a battery.

4. The sterilization system of claim 3, wherein said battery is rechargeable.

5. The sterilization system of claim 1, wherein said at least one bulb is a UV-C bulb.

6. An ultraviolet radiation system for a container comprising:
a container having a door capable of being opened, an insert, a front edge, a left wall, a right wall and an interior chamber;
at least one UV light source attached to either said right or left wall;
a power source; and
a switch that is automatically releasable.

7. The ultraviolet radiation system of claim 6 further comprising at least one sensor.

8. The ultraviolet radiation system of claim 7, wherein said at least one sensor is a pressure sensor.

9. The ultraviolet radiation system of claim 6 furtherer comprising a controller and a timer.

10. The ultraviolet radiation system of claim 9, wherein the switch energizes the timer when the door is opened, which allows the power source to illuminate the UV light source.

11. The ultraviolet radiation system of claim 6 further comprising a warning indicator and a safety indicator.

12. The ultraviolet radiation system of claim 6, wherein said power source is one of a direct wiring, a battery or a solar panel.

13. The ultraviolet radiation system of claim 6, wherein said ultraviolet radiation system is affixed to an outer surface of said container.

14. A method of operating the ultraviolet radiation system of claim 6 comprising:
opening said container;
releasing automatically said switch;
deactivating said UV light source; and
closing said container and activating said ultraviolet radiation system.

15. An ultraviolet radiation sterilization system for use with a drawer having an interior chamber wherein ultraviolet radiation occurs, wherein the ultraviolet radiation sterilization system comprises:
at least one UV-C bulb or tube;
a plurality of sensors to detect if said drawer is in an open position or a closed position, wherein ultraviolet illumination only occurs when said drawer is in the closed position; and
a power wire leading to a power supply.

16. The ultraviolet radiation sterilization system of claim 15, wherein said drawer is a cash drawer.

17. The ultraviolet radiation sterilization system of claim 15, wherein at least one of said plurality of sensors is a motion sensor.

18. The ultraviolet radiation sterilization system of claim 15, wherein at least one of said plurality of sensors is a pressure sensor.

19. The ultraviolet radiation sterilization system of claim 15 further comprising a timer.

20. The ultraviolet radiation sterilization system of claim 15 further comprising a warning indicator and a safety indicator.
